(19) United States
(12) Plant Patent Application Publication
(10) Pub. No.: US 2002/0174467 P1
(43) Pub. Date: Nov. 21, 2002

(54) Bidens plant named Golden Flame

(75) Inventor: Michael Helmet Unger, Rheinfelden (DE)

Correspondence Address: ROCKEY, MILNAMOW & KATZ, LTD., Two Prudential Plaza, 180 North Stetson, Suite 4700, Chicago, IL, 60601, US

(73) Assignee: Unger Breeding

(21) Appl. No.: 09/819,887

(22) Filed: Mar. 28, 2001

(51) Int. Cl. A01H 5/00

(52) U.S. Cl. PLT/263

(57) ABSTRACT

A new and distinct Bidens plant named Golden Flame, characterized by its bright yellow flowers, trailing habit, and medium green foliage.

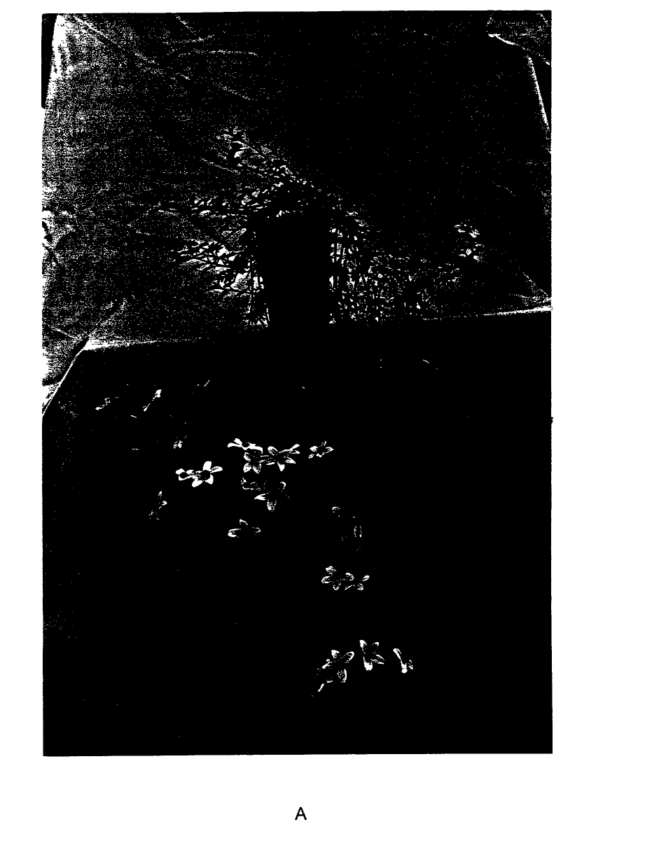

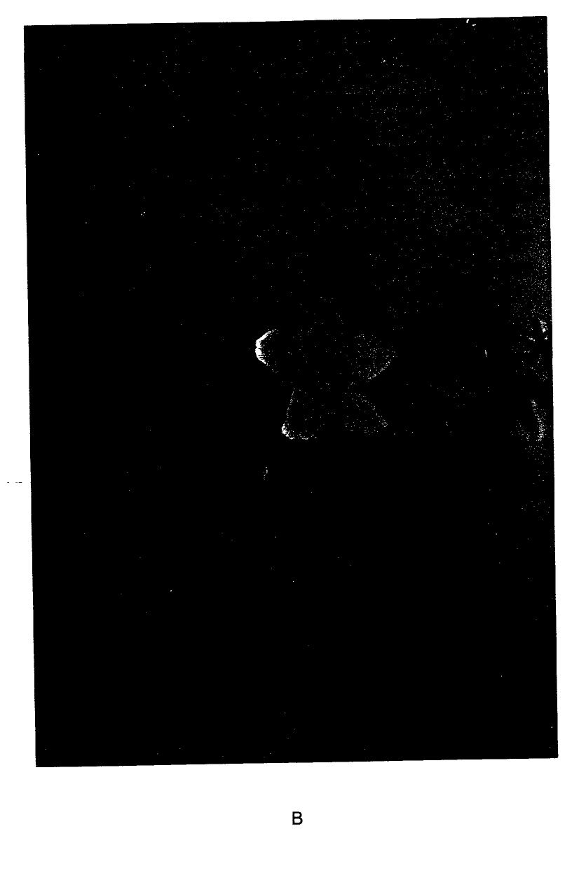

### BACKGROUND OF THE INVENTION

The present invention comprises a new and distinct Bidens plant, botanically known as Bidens feruifolia and hereinafter referred to by the cultivar name Golden Flame.

The new cultivar was developed by the inventor through a controlled breeding program during 1996, at Rheinfelden, Germany.

The female (seed) parent of Golden Flame was the commercially available Samsawa cultivar (not patented). The male (pollen) parent of Golden Flame was the commercially available Samsara (not patented).

Asexual reproduction of the new cultivar by terminal cuttings has demonstrated that the characteristics of the new cultivar as herein described are firmly fixed and retained through successive generations of such asexual propagation.

### SUMMARY OF THE INVENTION

It was found that the cultivar of the present invention:

(a) exhibits bright yellow flowers,

(b) forms medium green foliage,

(c) exhibits a good basal branching character and

(d) exhibits a trailing growth habit.

The new cultivar of the present invention can be compared to lnnbid (U.S. Plant Pat. No. 11,261). However, it is found that the new cultivar is more compact, has larger leaves and slightly larger flowers as detailed in TABLE A.

### BRIEF DESCRIPTION OF PHOTOGRAPHS

The accompanying photographs show as nearly true as it is reasonably possible to make the same in color illustrations of this type, typical flower and foliage characteristics of the new cultivar. The plants were grown for 15 weeks in a greenhouse at West Chicago, Ill., U.S.A.

Photo A illustrates the growth habit of Golden Flame at fifteen weeks after the planting of a rooted cutting.

Photo B is a close-up view of an individual flower.

### DETAILED DESCRIPTION

The Golden Flame cultivar has not been observed under all possible environmental conditions to date. Accordingly, it is possible that the phenotype may vary somewhat with variations in the environment, such as temperature, light intensity, and day length.

The chart used in the identification of colors described herein is the R.H.S. Colour Chart of The Royal Horticultural Society, London, England. The color values were determined on Feb. 15, 2001. The readings were taken between 10:00 and 11:00 a.m. under natural daylight conditions. The plants were produced from cuttings taken from stock plants and grown under greenhouse conditions comparable to those used in commercial practice while utilizing a soilless growth medium and maintaining temperatures of approximately 72° F. during the day and approximately 65° F. during the night. The following descriptions and measurements were taken from plants grown for 12 weeks from the planting of rooted cuttings.

Classification:

Botanical.—Bidens ferulifolia cultivar Golden Flame

Parentage:

Female parent.—Samsawa

Male Parent.—Samsara

Propagation:

Type cutting.—Terminal tip

Time to Initiate Roots.—Approximately 7-10 days.

Plant Description:

Habit of Growth.—Moderately vigorous with good basal branching. Pinching improves basal branching. A mature plant, 10-12 weeks after the planting of a rooted cutting, commonly measures approximately 11.4 cm in height, approximately 30 cm in diameter and 38.5 cm from top of pot to bottom of foliage.

Form.—Trailing

Lateral Stem.—Diameter: Approximately 2 mm. Texture: Covered with short soft hairs except at nodes where hairs are longer. Color: 146D with anthocyanin pigmentation of 183C. Internode length is approximately 4 cm.

Foliage.—Pinnatifid, opposite, with acute apex, cuneate base. Texture: Upper surface has sparse amount of short soft hairs, lower surface is smooth, with a few hairs along veins. Size: Approximately 3.2 cm in length and approximately 2.6 cm at widest point. Upper surface is 143A, lower surface is 146B. Venation: Upper surface is darker than 137A, lower surface is 137A. Petiole: Length—13 mm. Color—183C.

Flowering Description:

Flowering Habit.—Freely flowering.

Natural Flowering Season.—From spring through fall.

Inflorescence type.—Single, composite, persistent.

Bud.—9 mm in length, 5 mm in diameter.

Inflorescence diameter.—Approximately 2.7 cm

Ray florets.—Quantity: 5. Appearance: Smooth. Shape: Ovate. Length:1.5 cm. Width: 8 mm.

Color.—Upper surface, fully opened ray floret: Between 9A and 12A. Lower surface, fully opened ray floret: 7A with three prominent veins of 144C.

Capitulum.—Diameter is approximately 6 mm, depth is approximately 4 mm.

Disc florets.—Quantity: Approximately 28. Length: Approximately 5 mm. Diameter: Approximately 1 mm. Color: Closest to 14A.

Peduncle.—Length: Approximately 3.5 cm. Color: Closest to 144B.

Fragrance.—None

Reproductive Organs.—Androecium: Present on dics florets only. Anther color is 21B. Pollen color is 21A. Gynoecium: Present on ray and disc florets.

Seed Production: Seed production has not been observed.

Disease Resistance: Resistance to pathogens has not been observed.

I claim:

1. A new and distinct cultivar of Bidens ferulifolia named Golden Flame substantially as herein shown and described, which:
(a) exhibits bright yellow flowers,
(b) forms medium green foliage,
(c) exhibits a good basal branching character and
(d) exhibits a trailing growth habit.
TABLE A

| | GOLDEN | |
|---|---|---|
| CHARACTERISTIC | FLAME | INNBID |
| PLANT HEIGHT-CM | 11.4 | 16 |
| PLANT DIAMETER-CM | 30 | 45 |
| INTERNODE LENGTH-CM | 4 | 2 |
| PETIOLE LENGTH × DIAMETER-MM | 13 × 1 | 5 × 1 |
| LEAF LENGTH × WIDTH-CM | 3.2 × 2.6 | 2.4 × 1.3 |
| INFLORESCENCE DIAMETER-CM | 2.7 | 2.8 |
| # RAY FLORETS | 5 | 5 |
| RAY FLORET LENGTH × WIDTH-CM | 1.5 × .8 | 1.2 × .7 |
